(19) United States
(12) Patent Application Publication
(10) Pub. No.: US 2014/0061328 A1
(43) Pub. Date: Mar. 6, 2014

(54) INTERCHANGABLE LID AND BASE FOR AIR FRESHENER

(71) Applicant: American Covers, Inc., Draper, UT (US)

(72) Inventor: Bryce Haymond, Pleasant Groove, UT (US)

(21) Appl. No.: 13/837,918

(22) Filed: Mar. 15, 2013

Related U.S. Application Data
(60) Provisional application No. 61/694,906, filed on Aug. 30, 2012.

(51) Int. Cl. A61L 9/12; B65D 43/02

(52) U.S. Cl. CPC A61L 9/12; B65D 43/02
USPC 239/6; 215/228; 239/58

(57) ABSTRACT

An air freshener or container has a convertible and interchangeable lid/base that can form both a lid covering a jar and a base supporting a jar. In addition, the container or jar can contain water beads and a scented material.

### PRIORITY CLAIM(S)

Priority is claimed to U.S. Provisional Patent Application Ser. No. 61/694,906, filed Aug. 30, 2012, which is hereby incorporated herein by reference.

### BACKGROUND

1. Field of the Invention

The present invention relates generally to air fresheners and containers with interchangeable lids and bases.

2. Related Art

Air fresheners typically provide a desirable scent or fragrance, and can also provide an aesthetically pleasing appearance. Often, the air fresheners are sealed during shipment to prevent premature scent release, and/or to resist premature drying of the scent material. The development of aesthetically pleasing air fresheners is an ongoing endeavor.

### SUMMARY OF THE INVENTION

It has been recognized that it would be advantageous to develop an air freshener that is aesthetically pleasing. In addition, it has been recognized that it would be advantageous to develop an air freshener that provides an interesting and/or pleasing visual effect, particularly with light. In addition, it has been recognized that it would be advantageous to develop an air freshener and/or container that minimized parts, and/or that uses components for more than one purpose and in more than one location.

The invention provides a container with a jar having a cavity therein and a top opening. A convertible and interchangeable lid is removably disposed on the jar and covers the top opening of the jar, and is removably disposable under the jar. The lid is formed of or includes a flexible and resilient material. A crease is formed in the lid at an intermediate location between a top and a bottom thereof. The top of the lid is capable of being inverted about the crease to form an indentation, and defining a base converted from the lid. The lid is removable from the top opening of the jar, inverted to define the base, and placed underneath the jar with the indentation receiving at least a portion of the jar.

In accordance with a more detailed aspect of the invention, the container can be configured as an air freshener with a scent material disposed in the cavity of the jar. In addition, a plurality of water beads can be disposed in the cavity of the jar. The plurality of water beads can be or can include a water absorbing polymer capable of absorbing water and expanding. The plurality of water beads can be at least translucent when hydrated.

In addition, the invention provides an air freshener with a substantially spherical container including a substantially spherical lateral wall and a substantially spherical top wall and a flat bottom wall. The container includes a jar with the flat bottom wall and at least a portion of the spherical lateral wall. The jar has a cavity therein and a top opening. At least a portion of the jar is at least translucent. A plurality of water beads is disposed in the cavity of the jar. The plurality of water beads is or includes a water absorbing polymer capable of absorbing water and expanding, and is at least translucent when hydrated. A scent material is disposed in the cavity of the jar with the plurality of water beads. A convertible and interchangeable lid is removably disposed on the jar and covers the top opening of the jar, and is removably disposed under the jar and carrying the jar. The lid has a truncated, substantially spherical shaped shell forming the substantially spherical top wall and at least a portion of the spherical lateral wall of the container. The lid is substantially flush with the jar to form the substantially spherical container. The lid is formed of or includes a flexible and resilient material. The lid has a crease at an intermediate location between a top and a bottom thereof. The top of the lid is capable of being inverted about the crease to form a truncated, substantially spherical indentation, and define a base converted from the lid. The lid is removable from the top opening of the jar, inverted to define the base, and placed underneath the jar with the truncated, substantially spherical indentation receiving at least a portion of the substantially spherical lateral wall of the jar.

Furthermore, the invention provides a method for providing a desired fragrance that includes removing a lid from a jar. The lid and the jar form a substantially spherical container including a substantially spherical lateral wall and a substantially spherical top wall and a flat bottom wall. The jar forms the flat bottom wall and at least a portion of the spherical lateral wall. The jar has a cavity therein and a top opening. At least a portion of the jar is at least translucent. The lid has a truncated, substantially spherical shaped shell forming the substantially spherical top wall and at least a portion of the spherical lateral wall of the container. The lid is removably disposed on the jar and covers the top opening of the jar. The lid is formed of or includes a flexible and resilient material. The method includes adding water to a plurality of water beads disposed in the cavity of the jar. The plurality of water beads is or includes a water absorbing polymer capable of absorbing water and expanding, and is at least translucent when hydrated. The method includes adding a scent material into the cavity of the jar with the plurality of water beads. The method includes inverting a top of the lid about a crease at an intermediate location between the top and a bottom of the lid to form a base with the top forming a truncated, substantially spherical indentation. The method includes placing the base underneath the jar with the truncated, substantially spherical indentation receiving at least a portion of the substantially spherical lateral wall of the jar.

In accordance with a more detailed aspect of the invention, the method can include: removing the base from underneath the jar; inverting the top of the lid about the crease to form the lid with the top forming a truncated, substantially spherical shape; and replacing the lid on the jar.

### BRIEF DESCRIPTION OF THE DRAWINGS

Additional features and advantages of the invention will be apparent from the detailed description which follows, taken in conjunction with the accompanying drawings, which together illustrate, by way of example, features of the invention; and, wherein:

FIG. 1 is a front and top perspective view of an air freshener in accordance with an embodiment of the invention shown with a lid on a jar;

FIG. 2 is a front view of the air freshener of FIG. 1 shown with the lid on the jar and disposed on a support surface;

FIG. 3 is a front view of the air freshener of FIG. 1 shown with the lid removed from the jar and converted to a base with the jar thereon, and disposed on the support surface;

FIG. 4 is a side view of the air freshener of FIG. 1 shown with the jar disposed on the base;

FIG. 5 is a front and bottom perspective view of the air freshener of FIG. 1 shown with the lid on the base;

FIG. 6 is a bottom view of the air freshener of FIG. 1;

FIG. 7 is a top view of the air freshener of FIG. 1;

FIG. 8 is a side view of the air freshener of FIG. 1 shown with the lid disposed on the jar;

FIG. 9 is a front view of the air freshener of FIG. 1 shown with the lid removed from the jar;

FIG. 10a is a front view of the lid of the air freshener of FIG. 1;

FIG. 10b is front view of the lid of the air freshener of FIG. 1 converted into the base;

FIG. 11 is a front cross-sectional view of the air freshener of FIG. 1 shown with the lid removed from the base;

FIG. 12 is a front cross-sectional view of the air freshener of FIG. 1 taken along line 12 of FIG. 7 shown with the lid on the jar;

FIG. 13 is a front cross-sectional view of the jar of the air freshener of FIG. 3;

FIG. 14a is a front cross-sectional view of the lid of the air freshener of FIG. 1;

FIG. 14b is front cross-sectional view of the lid of the air freshener of FIG. 1 converted into the base; and

FIG. 15 is a front schematic view of the air freshener of FIG. 1 showing the lid being converted to a base.

Reference will now be made to the exemplary embodiments illustrated, and specific language will be used herein to describe the same. It will nevertheless be understood that no limitation of the scope of the invention is thereby intended.

### DETAILED DESCRIPTION OF EXAMPLE EMBODIMENT(S)

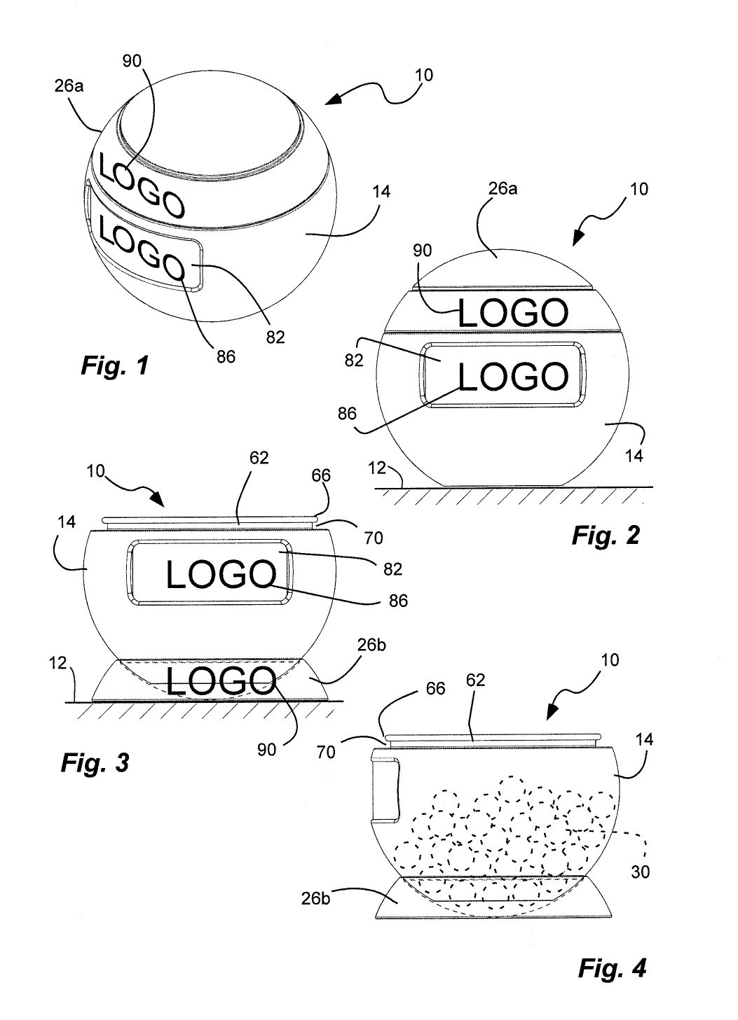

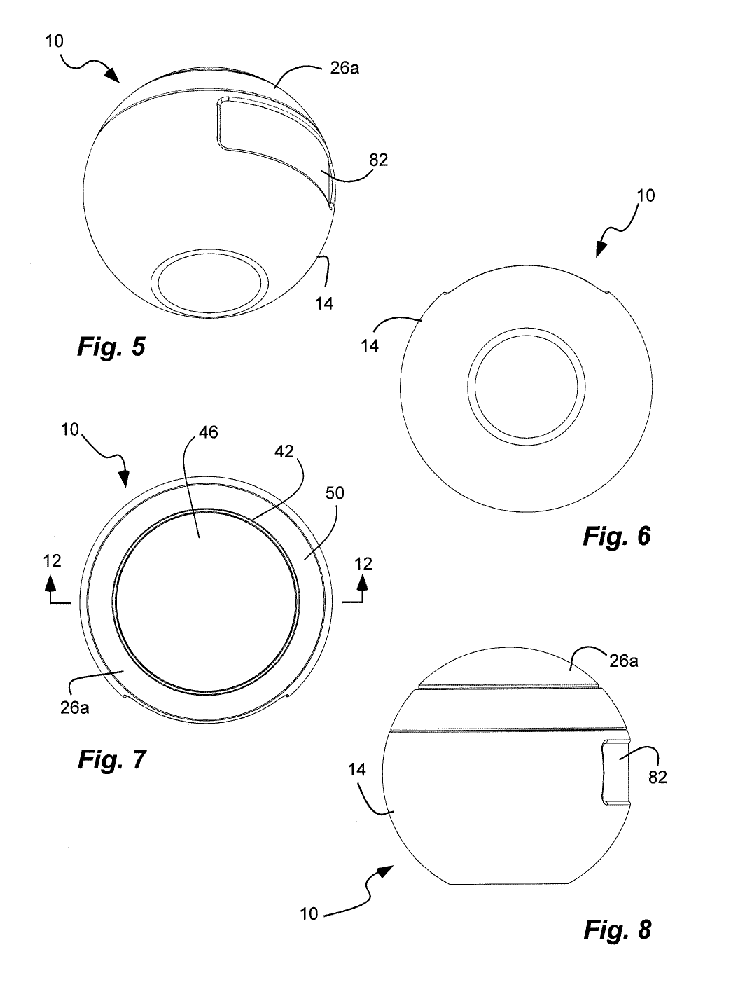

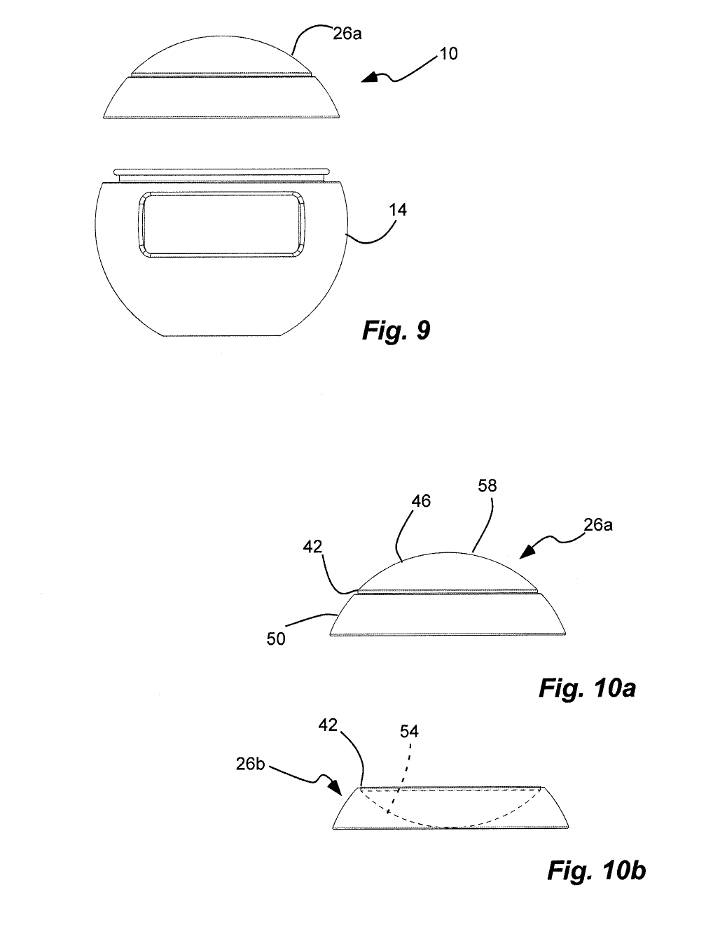

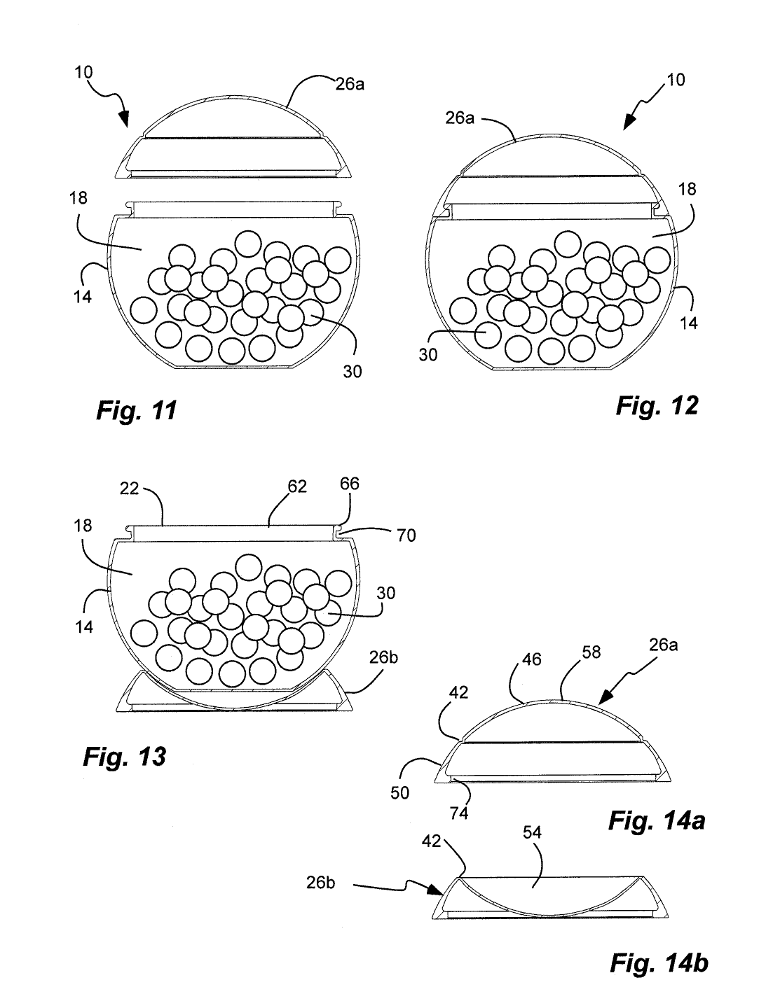

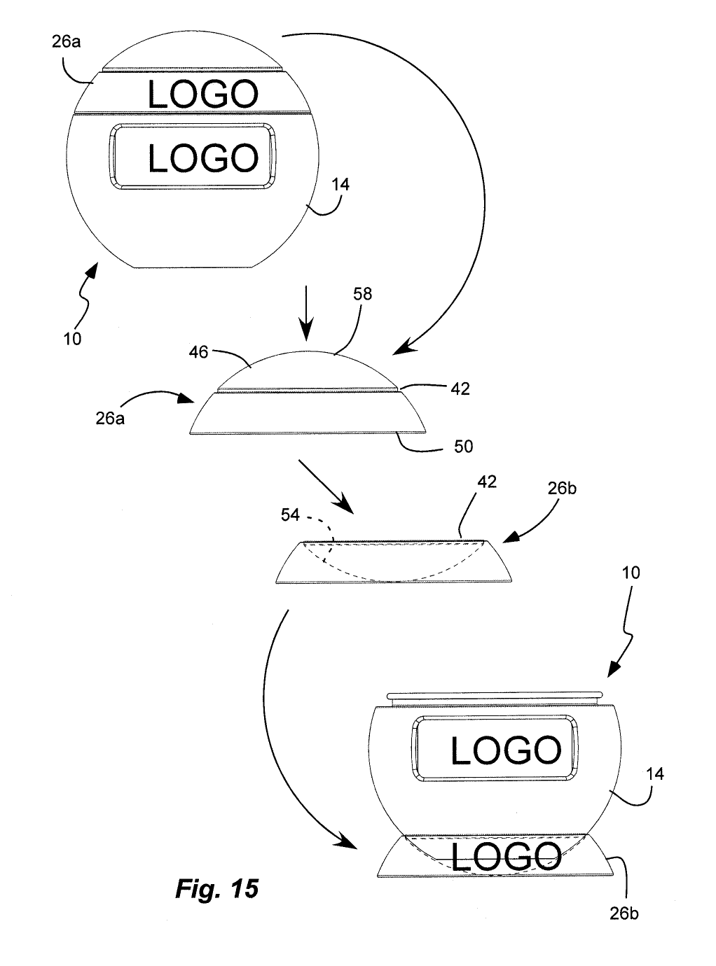

As illustrated in FIG. 1-15, an air freshener and/or a container, indicated generally at 10, in an example implementation in accordance with the invention is shown. The air freshener or container 10 can be disposed on a support surface 12, such as a table, counter, vanity, shelf, floor, ground, cup holder of a vehicle, etc. Thus, the air freshener can be used in an office, home, or vehicle. The air freshener can provide an aesthetically pleasing scent, fragrance, aroma and/or neutralizing agent. In addition, the air freshener can provide an aesthetically pleasing appearance, and/or interaction with light. Air fresheners are one example of a field that can benefit from the present invention. The terms “scent material,” “fragrance,” “scent,” and “aroma” are used interchangeably herein and are used broadly herein to refer to a material that has a scent that can be discernable or smelled, or even a neutralizing agent. Thus, the scent can be an ascertainable smell used to cover other scents, or a neutral agent that eliminates odors or provides a fresher atmosphere. The air freshener can be used in bathrooms, wash closets, vehicles, offices, bedrooms, etc.

The air freshener and/or container 10 can have a jar 14 with a cavity 18 therein and a top opening 22. The jar 14 can be formed of glass or plastic, and it or a portion thereof can be translucent or transparent. Thus, the jar or a portion thereof can be clear or transparent such that the contents thereof are clearly visible; or the jar or a portion thereof can be translucent, or cloudy, or colored, so that the contents may or may not be visible, but light is capable of passing therethrough. (The term transparent is used herein to mean substantially clear and visible therethrough; while the term translucent is used herein to mean substantially unclear but still transmissible to light, and may include cloudy, colored, misty, hazy, wavy, etc.) In one aspect, the container 10 can be a substantially spherical container including a substantially spherical lateral wall and a substantially spherical top wall and a flat bottom wall. The container 10 can have a substantially spherical or circular profile defining over 75 percent of a circle in one aspect, over 80 percent in another aspect, and over 90 percent in another aspect. The jar 14 can have or can form the flat bottom wall and at least a portion of the spherical lateral wall. The container and the jar can have other shapes, including for example, square, rectangular, triangular, oval, egg-shaped, gem-shaped, etc.

A plurality of water beads 30 can be disposed in the cavity 18 of the jar 14. The water beads can be provided dehydrated during shipping, and can be hydrated prior to use. Alternatively, the water beads can be hydrated prior to shipping and sealed inside the jar with an air-tight seal, such as a membrane of foil or the like, to prevent dehydration, evaporation, leakage, and diffusion of the scent prior to opening. The membrane can have indicia, such as marketing or branding, thereon. The plurality of water beads can be or can including a water absorbing polymer capable of absorbing water and expanding, such as such as a polyacrylamide (polymethylacrylate or sodium polyacrylate) or a superabsorbent polymer (SAP). The water beads can be at least translucent when hydrated. In addition, the water beads can be colored, the same or different colors. Such water beads are commercially available. The water beads can absorb water and can slowly release the water or moisture over time. Another light transmissive material, other than water beads can be used, such as plastic or glass beads or balls, a gel, a fiber, a woven or random fiber mat or disc, a liquid, an oil, etc.

A scent material can be disposed in the cavity of the jar with the plurality of water beads. The water beads can absorb the scent material, and can slowly release the scent material over time. The scent material can be or can include a scented oil. In addition, the scent material can be disposed in water or gel in the jar. After the water beads have fully or partially dehydrated and the scent is fully or partially evaporated, water can be re-added to the jar, and the water beads will be rehydrated to their full capacity again to continuously create a pleasing visual effect. In addition, the scent material can be re-added to the jar to continue the use as an air freshener.

In another aspect, the scent material can be disposed in another material, such as gel, oil, wax, etc, disposed in the cavity of the jar. Thus, the cavity of the jar can include the gel, oil or wax, with the scent material therein. Similarly, the material in the cavity of the jar can be transparent, translucent or opaque.

A convertible and interchangeable lid/base 26 (shown as lid 26a and base 26b) can be removably disposed on the jar 14 to cover the top opening of the jar (as a lid), and removably disposable under the jar (as a base). Thus, the lid/base 26 is reusable as the lid and/or the base; and can be repeatedly moved between the lid and base configurations. In addition, the base can be used as a holder or supporter for other spherical objects, such as baseballs, stress balls, etc., other items, such as paper clips, rubber bands, binder clips, etc. Thus, the reuse of the lid/base is environmentally friendly. The base can be removed from below the jar at any time and place over the jar as the lid in order to preserve and/or control the scent and prevent evaporation and prolong the use. The lid 26a can provide an air-tight seal with the jar 14. The lid/base (as the lid) can have a truncated, substantially spherical shaped shell forming the substantially spherical top wall and at least a portion of the spherical lateral wall of the container. The lid 26a can be substantially flush with the jar 14 to form the substantially spherical container.

The lid/base 26 can be formed of or can include a flexible and resilient material, such as rubber or silicon. The lid/base 26 can have a crease 42 at an intermediate location between a top 46 and a bottom 50 thereof. The crease 42 can be formed by an annular indentation or groove on one or both sides (inside and/or outside) of the lid 26 or shell thereof creating an area of reduced thickness with respect to the rest of the lid or shell. The top 46 of the lid/base can be capable of being inverted about the crease 42 to form a truncated, substantially spherical indentation 54, and defining the base 26b. Thus, the top 46 of the lid/base can be inverted between a convex protrusion 58 and a concave indentation 54. In use, the lid 26a can be removable from a top or top opening of the jar 14, the top 46 of the lid 26a can be inverted to define the base 26b, and placed underneath the jar 14. The truncated, substantially spherical indentation 54 can receive at least a portion of the substantially spherical lateral wall of the jar 14. The base 26b can be disposed on the support surface 12 and the material of the base can form a non-slip surface. In addition, the base 26b can be sized to have a width or diameter greater than a majority of the width or diameter of the jar to provide stability.

In addition, the container 10, or the lid 26 and the jar 14, can include a closure therebetween to secure the lid 26a to the jar 14 when the lid/base is configured as the lid and disposed on the jar. The closure can include interlocking annular lips on both the lid and the jar. The jar 14 can have an annular, upwardly protruding flange 62 extending from the top opening 22 of the jar; and an annular, outwardly protruding outer lip 66 extending radially outwardly from and beyond the flange 62, and defining and annular groove 70 between the outer lip, the flange and the jar. The lid 26a can have an annular, inwardly protruding inner lip 74 extending radially inwardly from the bottom opening of the lid/base. The inner lip 74 of the bottom opening of the lid can be expandable to stretch over the outer lip 66 of the jar 14 under an applied force to both remove the lid from the jar and place the lid on the jar, and being resilient to contract into the groove 70 to maintain the lid on the jar and resist removal thereof. It will be appreciated that the closure structure described above can be reversed with respect to the lid and jar.

In one aspect, the lid/base, or portion thereof, is opaque and has a color. In another aspect, the lid/base, or portion thereof, is translucent or transparent.

The plurality of water beads 30 can be translucent or transparent and can have multiple reflective surfaces when hydrated. As described above, the jar or a portion thereof can be translucent or transparent. The jar and the water beads can reflect and refract light, creating a unique and visually aesthetic appearance. In addition, the indentation 54 of the base 26b can be colored and can impart a color thereof to the light in the cavity, thus coloring the water beads. The beads can also be colored, or can include coloring.

The air freshener or container can also include an indentation 82 formed in the substantially spherical lateral wall of the jar. Indicia 86 can be disposed in the indentation. The indicia can include a logo or the like, and can be formed into the material of the container or jar, or can be applied by a sticker, etc. Indicia 90 can also be disposed on a lateral facing wall of the bottom of the lid/base. Thus, the indicia is visible in either configuration (lid or base). The indicia can be embossed or debossed. The indicia can be molded into the material of the lid/base, or can be printed (screen or pad) thereon. The indicia can include one or more labels disposed on the lateral wall of the jar, in the indentation of the jar, and or on the lid and/or base. The indicia can include a logo, icon, corporate message, brand, etc.

Furthermore, the container, jar and/or lid/base can be decorated, such as with a screen print, silk screen print, etc.

A method of using the air freshener 10 described above, and for providing a desired fragrance, includes removing a lid 26a from a jar 14, the lid and the jar forming a substantially spherical container 10 including a substantially spherical lateral wall and a substantially spherical top wall and a flat bottom wall. The jar forms the flat bottom wall and at least a portion of the spherical lateral wall. The jar has a cavity 18 therein and a top opening 22. At least a portion of the jar is at least translucent. The lid has a truncated, substantially spherical shaped shell forming the substantially spherical top wall and at least a portion of the spherical lateral wall of the container. The lid is removably disposed on the jar and covers the top opening of the jar. The lid is formed of or includes a flexible and resilient material. Water is added to a plurality of water beads 30 disposed in the cavity of the jar. The plurality of water beads is or includes a water absorbing polymer capable of absorbing water and expanding. The water beads are at least translucent when hydrated. A scent material is added into the cavity of the jar with the plurality of water beads. A top 46 of the lid is inverted about a crease 42 at an intermediate location between the top and a bottom 50 of the lid to form a base 26b with the top forming a truncated, substantially spherical indentation 54. The base is placed underneath the jar with the truncated, substantially spherical indentation receiving at least a portion of the substantially spherical lateral wall of the jar.

In addition, the base can be removed from underneath the jar. The top 46 of the lid can be inverted about the crease to form the lid with the top forming a truncated, substantially spherical shape 58. The lid 26a can be replaced on the jar.

In accordance with another aspect of the invention, a light source can be associated with the container. The light source can be an LED electrically coupled to a battery. In one aspect, the light source can be disposed in the bottom of the cavity of the jar to emit light through the plurality of water beads and the jar. In another aspect, the light source can be carried by the lid, such as in a cavity thereof, above the water beads. In another aspect, the light source can be carried by the base, such as in a cavity thereof; and the base can have one or more apertures through which light can pass into the jar and the plurality of water beads. In another aspect, the light source can be suspended in the water beads. In another aspect, the light source can be carried by the jar, on an exterior thereof.

In accordance with another aspect of the invention, the light transmissive material or the water beads can further include another distinct material interspersed therein, such as glass or plastic beads, or other inclusions. The another material or inclusions can be a different color, shape, size, etc. The another material or inclusions can include a gel, a liquid, plastic beads, etc. The inclusions can be shiny or have a mirrored or reflective surface. The inclusions can include a sparkly grit. The another material or inclusions can be interspersed throughout the light transmissive material or water beads, or can be disposed above or below.

While the forgoing examples are illustrative of the principles of the present invention in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the invention be limited, except as by the claims set forth below.

1. A container device, comprising:
a) a jar having a cavity therein and a top opening;
b) a convertible and interchangeable lid removably disposed on the jar and covering the top opening of the jar, and removably disposable under the jar;
c) the lid being formed of or including a flexible and resilient material;
d) a crease formed in the lid at an intermediate location between a top and a bottom thereof;
e) the top of the lid being capable of being inverted about the crease to form an indentation and defining a base converted from the lid; and
f) the lid being removable from the top opening of the jar, inverted to define the base, and placed underneath the jar with the indentation receiving at least a portion of the jar.

2. A device in accordance with claim 1, further comprising:
a scent material disposed in the cavity of the jar.

3. A device in accordance with claim 2, further comprising:
a plurality of water beads disposed in the cavity of the jar, the plurality of water beads being or including a water absorbing polymer capable of absorbing water and expanding, and being at least translucent when hydrated.

4. A device in accordance with claim 1, further comprising:
a) a light transmissive material disposed in the cavity of the jar, the light transmissive material being translucent or transparent and having multiple reflective surfaces;
b) at least a portion of the jar being translucent or transparent; and
c) the at least a portion of the jar and the light transmissive material reflecting and refracting light, and the indentation of the base imparting a color thereof to the light.

5. A device in accordance with claim 1, further comprising:
a plurality of water beads disposed in the cavity of the jar, the plurality of water beads being or including a water absorbing polymer capable of absorbing water and expanding, and being at least translucent when hydrated.

6. A device in accordance with claim 5, further comprising:
a scent material disposed in the cavity of the jar.

7. A device in accordance with claim 1, further comprising:
the lid having a truncated, substantially spherical shaped shell forming a substantially spherical top wall and at least a portion of a spherical lateral wall.

8. A device in accordance with claim 1, further comprising:
a) an indentation formed in the substantially spherical lateral wall of the jar; and
b) indicia disposed in the indentation.

9. A device in accordance with claim 1, further comprising:
indicia disposed on a lateral facing wall of the bottom of the lid or the base.

10. A device in accordance with claim 1, further comprising:
a) a bottom opening in the lid;
b) a closure between the lid and the jar to secure the lid to the jar when the lid is configured as the lid, and including:
i) an annular, upwardly protruding flange extending from the top opening of the jar;
ii) an annular, outwardly protruding outer lip extending radially outwardly from and beyond the flange, and defining and annular groove between the outer lip, the flange and the jar;
iii) an annular, inwardly protruding inner lip extending radially inwardly from the bottom opening of the lid; and
iv) the inner lip of the bottom opening of the lid/base being expandable to stretch over the outer lip under an applied force to both remove the lid from the jar and place the lid on the jar, and being resilient to contract into the groove to maintain the lid on the jar and resist removal thereof.

11. A device in accordance with claim 1, further comprising:
a) the plurality of water beads being translucent or transparent and having multiple reflective surfaces when hydrated;
b) the least a portion of the jar being translucent or transparent; and
c) the at least a portion of the jar and the plurality of water beads reflecting and refracting light, and the indentation of the base imparting a color thereof to the light.

12. An air freshener device, comprising:
a) a substantially spherical container including a substantially spherical lateral wall and a substantially spherical top wall and a flat bottom wall, and including a jar with the flat bottom wall and at least a portion of the spherical lateral wall, the jar having a cavity therein and a top opening;
b) at least a portion of the jar being at least translucent;
c) a plurality of water beads disposed in the cavity of the jar, the plurality of water beads being or including a water absorbing polymer capable of absorbing water and expanding, and being at least translucent when hydrated;
d) a scent material disposed in the cavity of the jar with the plurality of water beads;
e) a convertible and interchangeable lid removably disposed on the jar and covering the top opening of the jar, and removably disposed under the jar and carrying the jar;
f) the lid having a truncated, substantially spherical shaped shell forming the substantially spherical top wall and at least a portion of the spherical lateral wall of the container and being substantially flush with the jar to form the substantially spherical container;
g) the lid being formed of or including a flexible and resilient material;
h) the lid having a crease at an intermediate location between a top and a bottom thereof;
i) the top of the lid being capable of being inverted about the crease to form a truncated, substantially spherical indentation and defining a base converted from the lid; and
i) the lid being removable from the top opening of the jar, inverted to define the base, and placed underneath the jar with the truncated, substantially spherical indentation receiving at least a portion of the substantially spherical lateral wall of the jar.

13. A device in accordance with claim 12, further comprising:
a) an indentation formed in the substantially spherical lateral wall of the jar; and
b) indicia disposed in the indentation.

14. A device in accordance with claim 12, further comprising:
indicia disposed on a lateral facing wall of the bottom of the lid or the base.

15. A device in accordance with claim 12, further comprising:
a) a bottom opening in the lid;
b) a closure between the lid and the jar to secure the lid to the jar when the lid is configured as the lid, and including:
i) an annular, upwardly protruding flange extending from the top opening of the jar;
ii) an annular, outwardly protruding outer lip extending radially outwardly from and beyond the flange, and defining and annular groove between the outer lip, the flange and the jar;
iii) an annular, inwardly protruding inner lip extending radially inwardly from the bottom opening of the lid; and
iv) the inner lip of the bottom opening of the lid/base being expandable to stretch over the outer lip under an applied force to both remove the lid from the jar and place the lid on the jar, and being resilient to contract into the groove to maintain the lid on the jar and resist removal thereof.

16. A device in accordance with claim 12, further comprising:
a) the plurality of water beads being translucent or transparent and having multiple reflective surfaces when hydrated;
b) the least a portion of the jar being translucent or transparent; and
c) the at least a portion of the jar and the plurality of water beads reflecting and refracting light, and the indentation of the base imparting a color thereof to the light.

17. A method for providing a desired fragrance, comprising:
a) removing a lid from a jar, the lid and the jar forming a substantially spherical container including a substantially spherical lateral wall and a substantially spherical top wall and a flat bottom wall, the jar forming the flat bottom wall and at least a portion of the spherical lateral wall, the jar having a cavity therein and a top opening, at least a portion of the jar being at least translucent, the lid having a truncated, substantially spherical shaped shell forming the substantially spherical top wall and at least a portion of the spherical lateral wall of the container, the lid being removably disposed on the jar and covering the top opening of the jar, the lid being formed of or including a flexible and resilient material;
b) adding water to a plurality of water beads disposed in the cavity of the jar, the plurality of water beads being or including a water absorbing polymer capable of absorbing water and expanding, and being at least translucent when hydrated;
c) adding a scent material into the cavity of the jar with the plurality of water beads;
d) inverting a top of the lid about a crease at an intermediate location between the top and a bottom of the lid to form a base with the top forming a truncated, substantially spherical indentation; and
e) placing the base underneath the jar with the truncated, substantially spherical indentation receiving at least a portion of the substantially spherical lateral wall of the jar.

18. A method in accordance with claim 17, further comprising:
a) removing the base from underneath the jar;
b) inverting the top of the lid about the crease to form the lid with the top forming a truncated, substantially spherical shape; and
c) replacing the lid on the jar.
